(19) United States
(12) Patent Application Publication
(10) Pub. No.: US 2017/0334830 A1
(43) Pub. Date: Nov. 23, 2017

(54) METHOD OF REARRANGEMENT OF THE MOLECULAR STRUCTURE OF ISOMERS OF TEXANOL

(71) Applicant: RUNTAI CHEMICAL CO., LTD., JIANGSU (CN)

(72) Inventors: NING YU, JIANGSU (CN); SHIYUAN ZHANG, JIANGSU (CN)

(73) Assignee: RUNTAI CHEMICAL CO., LTD., JIANGSU (CN)

(21) Appl. No.: 15/235,122

(22) Filed: Aug. 12, 2016

(30) Foreign Application Priority Data
May 20, 2016 (CN) 201610336252.0

(51) Int. Cl. C07C 69/003; C07C 67/02

(52) U.S. Cl. CPC C07C 69/003; C07C 67/02

(57) ABSTRACT

Disclosed is a method of rearrangement of the molecular structure of isomers of TEXANOL, characterized in that two isomers in TEXANOL is subjected to rearrangement of the molecular structure at a certain temperature and pressure, and 2,2,4-trimethyl-1,3-pentanediol-3-isobutyrate and 2,2,4-trimethyl-1,3-pentanediol-1-isobutyrate in TEXANOL following the rearrangement are in a mass ratio of 31.81-38.65:60.80-67.65. The process of the present invention is simple, no catalyst and other aids are required, and the content of the primary ester in TEXANOL can be significantly increased, so as to improve the volatilization rate of TEXANOL in a paint film, thereby reducing the drying time of the paint film and increasing the number of scrubbing of the paint film, such that the construction conditions of aqueous acrylic paints for exterior walls become relaxed, and the occurrence of floating, whitening, efflorescence, and cracking of the paint film due to water absorption by the non-dried or partly dried paint film during moist or rainy periods is avoided.

### CROSS-REFERENCE TO RELATED APPLICATION

This application claims the priority benefit of China application serial no. 201610336252.0, filed on May 20, 2016. The entirety of the above-mentioned patent application is hereby incorporated by reference herein and made a part of this specification.

### BACKGROUND OF THE INVENTION

### 1. Field of the Invention

The present invention relates to the technical field of the chemical industry, and more particularly to a method of rearrangement of the molecular structure of isomers of TEXANOL.

### 2. Description of the Related Art

TEXANOL (alcohol ester-12, Texanol ester alcohol) has a chemical name of 2,2,4-trimethyl-1,3-pentanediol monoisobutyrate. TEXANOL is mainly used as a film-forming aid for latex paint, and can effectively reduce minimum film forming temperature of latex paint and enhance properties of paint film. TEXANOL does not hydrolyze in a general basic medium and has good storage stability. The component analysis of TEXANOL by gas chromatography indicates that TEXANOL is essentially consisted of two isomers, 2,2,4-trimethyl-1,3-pentanediol-3-isobutyrate and 2,2,4-trimethyl-1,3-pentanediol-1-isobutyrate, the following are respective structural formula:

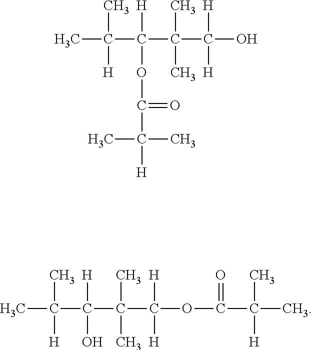

Some paint manufacturers have raised the question of thermal stability of the composition of the isomers of TEXANOL, and Hu Jingliang et al from GUANGDONG TEST CENTER OF PRODUCT QUALITY SUPERVISION (CEST) in China have carried out experimental studies on thermal stability of TEXANOL, and have found that under pyrolysis conditions (750° C., 10S), a resin in a paint film of a latex paint has been essentially pyrolyzed into fragment ions, but the peak of TEXANOL at a high abundance is present, indicating that the two isomers of TEXANOL have very high thermal stability. In addition, our researches have shown that when film of an aqueous acrylic emulsion paint is formed, TEXANOL in the paint film volatilizes along with water, generally it requires 7 days for completion. Only after TEXANOL in the paint film fully or essentially fully volatilized, the paint film can achieve basic properties and thus have the application value. Thus, how to speed up the volatilization of TEXANOL in the paint film is a technical problem that needs to resolve urgently.

### SUMMARY OF THE INVENTION

An object of the present invention is to provide a method of rearrangement of the molecular structure of isomers of TEXANOL. By using this method, the content of the primary ester in TEXANOL can be significantly increased, so that the volatilization rate of TEXANOL in the paint film is improved, thereby reducing the drying time of the paint film and increasing the scrub resistance of the paint film.

The technical solution adopted by the present invention is as follows.

A method of rearrangement of the molecular structure of isomers of TEXANOL, TEXANOL comprises of two isomers, 2,2,4-trimethyl-1,3-pentanediol-3-isobutyrate and 2,2,4-trimethyl-1,3-pentanediol-1-isobutyrate, in a mass ratio of 37.32-40.12:59.33-62.24, wherein the two isomers in TEXANOL is subjected to rearrangement of the molecular structure at a certain temperature and pressure, and 2,2,4-trimethyl-1,3-pentanediol-3-isobutyrate and 2,2,4-trimethyl-1,3-pentanediol-1-isobutyrate in TEXANOL following the rearrangement are in a mass ratio of 31.81-38.65:60.80-67.65.

Further, the temperature is 150-250° C. and the pressure is 0.1-2.0 MPa.

Further, the pressure is maintained for 0.25-5 h.

The advantages of the present invention are that: in the process, no catalyst or other aids are required, and the content of the primary ester in TEXANOL can be significantly increased, so as to increase the volatilization rate of TEXANOL in the paint film, thereby reducing the drying time of the paint film and increasing scrub resistance of the paint film, so that the construction condition requirement of aqueous acrylic paints for exterior walls is wided, and particularly the construction for the Yangtze river valley in the plum-rain season and for the southern region of China in the rainy season becomes relatively easy. The occurrence of floating, whitening, efflorescence, and cracking of the paint film due to water absorption by the non-dried or partly dried paint film during moist or rainy periods is avoided.

### DETAILED DESCRIPTION OF THE PREFERRED EMBODIMENTS

### Example 1

1700 g TEXANOL (having a content of 99.45%) with 2,2,4-trimethyl-1,3-pentanediol-3-isobutyrate and 2,2,4-trimethyl-1,3-pentanediol-1-isobutyrate being in a mass ratio of 40.12:59.33, was charged into a WHFS-3-type reactor. The reactor was controlled at a temperature of 160-165° C. and at a pressure of 0.3 MPa and maintained at this temperature and pressure for 5 h. The experimental results show that 2,2,4-trimethyl-1,3-pentanediol-3-isobutyrate and 2,2,4-trimethyl-1,3-pentanediol-1-isobutyrate are in a mass ratio of 38.65:60.80.

### Example 2

1700 g TEXANOL (having a content of 99.45%) with 2,2,4-trimethyl-1,3-pentanediol-3-isobutyrate and 2,2,4-trimethyl-1,3-pentanediol-1-isobutyrate being in a mass ratio of 40.12:59.33, was charged into a WHFS-3-type reactor. The reactor was controlled at a temperature of 160-165° C. and at a pressure of 0.6 MPa and maintained at this temperature and pressure for 0.25 h. The experimental results show that 2,2,4-trimethyl-1,3-pentanediol-3-isobutyrate and 2,2,4-trimethyl-1,3-pentanediol-1-isobutyrate are in a mass ratio of 38.05:61.40.

### Example 3

1500 g TEXANOL (having a content of 99.40%) with 2,2,4-trimethyl-1,3-pentanediol-3-isobutyrate and 2,2,4-trimethyl-1,3-pentanediol-1-isobutyrate being in a mass ratio of 38.12:61.28, was charged into a WHFS-3-type reactor. The reactor was controlled at a temperature of 170-180° C. and at a pressure of 1.6 MPa and maintained at this temperature and pressure for 20 min. The experimental results show that 2,2,4-trimethyl-1,3-pentanediol-3-isobutyrate and 2,2,4-trimethyl-1,3-pentanediol-1-isobutyrate are in a mass ratio of 35.01:64.39.

### Example 4

1500 g TEXANOL (having a content of 99.34%) with 2,2,4-trimethyl-1,3-pentanediol-3-isobutyrate and 2,2,4-trimethyl-1,3-pentanediol-1-isobutyrate being in a mass ratio of 38.12:61.22, was charged into a WHFS-3-type reactor. The reactor was controlled at a temperature of 170-180° C. and at a pressure of 0.8 MPa and maintained at this temperature and pressure for 35 min. The experimental results show that 2,2,4-trimethyl-1,3-pentanediol-3-isobutyrate and 2,2,4-trimethyl-1,3-pentanediol-1-isobutyrate are in a mass ratio of 33.11:66.23.

### Example 5

1500 g TEXANOL (having a content of 99.56%) with 2,2,4-trimethyl-1,3-pentanediol-3-isobutyrate and 2,2,4-trimethyl-1,3-pentanediol-1-isobutyrate being in a mass ratio of 37.32:62.24, was charged into a WHFS-3-type reactor. The reactor was controlled at a temperature of 190-210° C. and at a pressure of 1.8 MPa and maintained at this temperature and pressure for 45 min. The experimental results show that 2,2,4-trimethyl-1,3-pentanediol-3-isobutyrate and 2,2,4-trimethyl-1,3-pentanediol-1-isobutyrate are in a mass ratio of 31.91:67.65.

### Example 6

1500 g TEXANOL (having a content of 99.37%) with 2,2,4-trimethyl-1,3-pentanediol-3-isobutyrate and 2,2,4-trimethyl-1,3-pentanediol-1-isobutyrate being in a mass ratio of 38.52:60.85, was charged into a WHFS-3-type reactor. The reactor was controlled at a temperature of 150-155° C. and at a pressure of 2.0 MPa and maintained at this temperature and pressure for 2 h. The experimental results show that 2,2,4-trimethyl-1,3-pentanediol-3-isobutyrate and 2,2,4-trimethyl-1,3-pentanediol-1-isobutyrate are in a mass ratio of 31.81:67.56.

### Example 7

1500 g TEXANOL (having a content of 99.23%) with 2,2,4-trimethyl-1,3-pentanediol-3-isobutyrate and 2,2,4-trimethyl-1,3-pentanediol-1-isobutyrate being in a mass ratio of 37.56:61.67, was charged into a WHFS-3-type reactor. The reactor was controlled at a temperature of 215-250° C. and at a pressure of 0.1 MPa and maintained at this temperature and pressure for 3 h. The experimental results show that 2,2,4-trimethyl-1,3-pentanediol-3-isobutyrate and 2,2,4-trimethyl-1,3-pentanediol-1-isobutyrate are in a mass ratio of 32.27:66.96.

What is claimed is:

1. A method of rearrangement of the molecular structure of isomers of TEXANOL, TEXANOL comprises of two isomers, 2,2,4-trimethyl-1,3-pentanediol-3-isobutyrate and 2,2,4-trimethyl-1,3-pentanediol-1-isobutyrate, in a mass ratio of 37.32-40.12:59.33-62.24, characterized in that the two isomers in TEXANOL is subjected to rearrangement of the molecular structure at a certain temperature and pressure, and 2,2,4-trimethyl-1,3-pentanediol-3-isobutyrate and 2,2,4-trimethyl-1,3-pentanediol-1-isobutyrate in TEXANOL following the rearrangement are in a mass ratio of 31.81-38.65:60.80-67.65.

2. The method of rearrangement of the molecular structure of isomers of TEXANOL of claim 1, wherein the temperature is 150-250° C. and the pressure is 0.1-2.0 MPa.

3. The method of rearrangement of the molecular structure of isomers of TEXANOL of claim 1, wherein the pressure is maintained for 0.25-5 h.

4. The method of rearrangement of the molecular structure of isomers of TEXANOL of claim 2, wherein the pressure is maintained for 0.25-5 h.
